(19) United States
(12) Patent Application Publication
(10) Pub. No.: US 2019/0108899 A1
(43) Pub. Date: Apr. 11, 2019

(54) METHOD FOR IMPROVING HEALTH INFORMATION COLLECTION AND HEALTH-RELATED RECOMMENDATIONS THROUGH DATA COMPARTMENTALIZATION

(71) Applicant: Michael Robkin, Woodland Hills, CA (US)

(72) Inventor: Michael Robkin, Woodland Hills, CA (US)

(21) Appl. No.: 16/157,020

(22) Filed: Oct. 10, 2018

Related U.S. Application Data
(60) Provisional application No. 62/570,598, filed on Oct. 10, 2017.

(51) Int. Cl. G16H 10/60; G06N 5/02

(52) U.S. Cl. CPC G16H 10/60; G06N 5/025

(57) ABSTRACT

Health-related recommendations for an individual may be made by a system utilizing electronic health care records (EHR) and private data of or regarding the individual excluded from the EHR. EHR may be provided to a computer system which does not provide information for storage as part of an EHR. The provided EHR and the private data may be provided to a rules engine for determining the health-related recommendations.

### CROSS REFERENCE TO RELATED APPLICATION

This application claims the benefit of the filing date of U.S. Provisional Patent Application No. 62/570,598, filed on Oct. 10, 2017, the disclosure of which is incorporated by reference herein.

### BACKGROUND OF THE INVENTION

The present invention relates generally to generation of health care related recommendations, and more particularly to generation of health care related recommendations based on electronic health care records (EHR) and non-EHR private information.

A medical doctor may base patient therapeutical recommendations on information regarding the patient known to the doctor. This information may include information relating to the patient's general health, habits, past medical care, and other information. Often times, the information may be stored electronically, and is often termed Electronic Health Care Records (EHR), and health-related recommendations by the doctor may be based on the patients EHR.

The provision of appropriate health care, important as it is, may be fraught with difficulties. There are vagaries and differences between different people. There is a possible lack of a complete concrete understanding of all of the operations of the human body, and reaction of the human body to various drugs, substances, and regimens. Compounding all of this, medical doctors and similar health care professionals may face difficulties in eliciting accurate and/or complete information from patients.

Patients may be simply uncomfortable or unwilling to provide their doctor pertinent information. The cause may be embarrassment, an understanding that information provided to medical doctors will be stored as EHR and the EHR may be later legally obtained by insurance companies or other entities to bad effect to the patient, a recognition that the EHR may be available to parents, caregivers, relatives, employers, co-workers, business associates or compromised by bad actors, or a variety of rational or irrational reasons.

Some pertinent information may not be disclosed to the doctor and therefore not in the EHR. Indeed, some pertinent information disclosed to the doctor may even be false, therefore contradicting the information of the EHR. This pertinent information may be of importance in making recommendations as to possible lifestyle changes by the patient, in prescribing pharmaceuticals, or a host of other recommendations. Unfortunately, the root causes of why patients do not provide their medical doctors proper information for purposes of medical care may be difficult to address.

### BRIEF SUMMARY OF THE INVENTION

In some embodiments a system includes: a first memory storing electronic health care records (EHR) for a plurality of individuals; a first processor coupled to the first memory, the first processor configured to execute instructions of a rules engine associated with the first processor for determining a health-related recommendation for a particular individual of the plurality of individuals based on at least some of the EHR; at least one second memory storing private information for the particular individual of the plurality of individuals, the at least one second memory not readable by the first processor; and a second processor coupled to the second memory, the second processor configured to request and receive from the first processor the EHR for the particular individual and to execute instructions of a rules engine associated with the second processor for determining a health-related recommendation based on at least some of the EHR and at least some of the private information for the particular individual.

In some embodiments a method comprises: storing electronic health care records (EHR) for a plurality of individuals; executing instructions, by a first system, of a rules engine associated with the first system for determining a first health-related recommendation for a particular individual based on at least some the EHR; transmitting at least some of the EHR for the particular individual to a third party system, the third party system storing private information for the particular individual, at least some of the private information not being information included in the EHR; and executing instructions, by the third party system, of a rules engine associated with the third party system for determining a second health-related recommendation for the particular individual based on at least some the EHR and at least some of the private information for the particular information.

In some embodiments the method further comprises transmitting information of the second health-related recommendation to the first system.

In some embodiments the method further comprises providing information of the first health-related recommendation and the second health-related recommendation to the particular individual, without providing information of the second health-related recommendation to the first system.

In some embodiments the at least some of the EHR transmitted to the third party system comprises information of the first health-related recommendation. In some embodiments the method further comprises comparing, by the third party system, information of the first health-related recommendation and the second health-related recommendation.

In some embodiments the EHR for the particular individual includes an identification of the third party system.

In some embodiments the EHR for the particular individual does not include an identification of the third party system. In some embodiments the EHR for the particular individual includes a key for use in determining if a third party requestor should be allowed access to the EHR for the particular individual.

In some embodiments the rules engine associated with the first processor and the rules engine associated with the third party system are the same rules engine.

In some embodiments the rules engine associated with the first processor and the rules engine associated with the third party system implement the same rules.

In some embodiments the rules engine associated with the first processor and the rules engine associated with the third party system implement different rules.

In some embodiments a method comprises: storing electronic health care records (EHR) for a plurality of individuals; executing instructions, by a first system, of a rules engine for determining a first health-related recommendation for a particular individual based on at least some the EHR; transmitting options for the first health-related recommendation to a third party system, the third party system storing private information for the particular individual, at least some of the private information not being information included in the EHR; and executing instructions, by the third party system, of the rules engine based on at least some of the private information to determine whether options for the first health-related recommendation are contraindicated or re-prioritized by the private information.

In some embodiments the method further comprises determining that at least some options for the first treatment program are contraindicated by the private information.

In some embodiments the method further comprises transmitting results of the determination whether options for the first treatment program are contraindicated by the private information to the first system.

These and other aspects of the invention are more fully comprehended upon review of this disclosure.

### BRIEF DESCRIPTION OF THE FIGURES

FIG. 1 is a block diagram of a system in accordance with aspects of the invention.

FIG. 2 is a flow diagram of a process for creating a rules engine.

FIG. 3 is a flow diagram of a process for determining a health related recommendation using EHR and private data in accordance with aspects of the invention.

FIG. 4 is a block diagram showing data flow for a process for determining a health related recommendation using EHR and private data in accordance with aspects of the invention.

### DETAILED DESCRIPTION

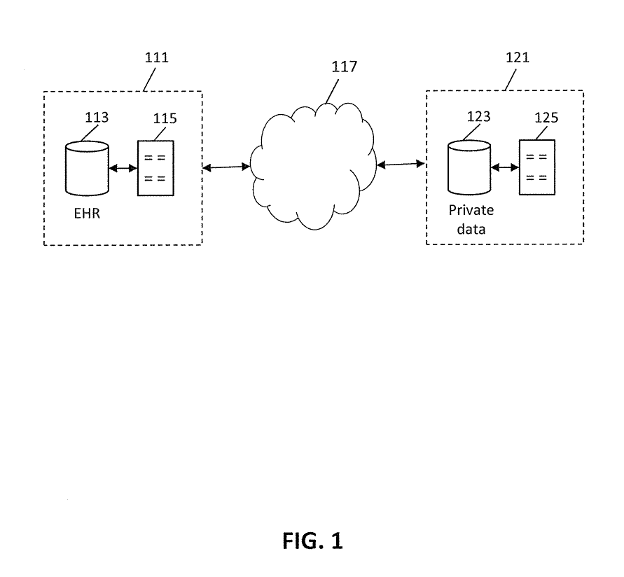

FIG. 1 is a block diagram of a system in accordance with aspects of the invention. The system includes a first computer system 111 of a medical provider or associated with a medical provider and a second computer system 121 of a third party. In various embodiments the third party may be, for example, a mental health professional, a social worker, or a care coordinator.

The first system includes a first memory 113 and a first computer 115, including at least one processor. The first memory 113 stores electronic health records (EHR), for example for a plurality of individuals, who may also be called patients from time-to-time herein. In some embodiments EHR is data about or concerning patients. In some embodiments EHR is comprised of medical, demographic, health, and social information stored in a computer system used by health care professionals. In some embodiments EHR does not include such information stored in computer system of a mental health professional. Data stored in the memory may be considered electronic medical records as described by laws, regulation, and professional practice. Data may also be other data such as doctor's practice notes, general advice, and reference material not considered to belong to the medical record of a patient. EHR may be regulated by government agencies, certified by government or professional organizations, or neither.

In some embodiments the first computer is configured to execute program instructions of a rules engine, using at least some of the EHR. The rules engine may provide clinical decision support (CDS) and may implement or be part of a CDS system or process. The rules engine may determine health-related recommendations for a patient based on rules of the rules engine and the EHR. For example, the rules engine may determine pharmaceutical recommendations for treatment of disease, lifestyle change recommendations for health-related reasons, or other health-related recommendations. In some embodiments the health-related recommendations are also considered EHR, and in some embodiments also stored in the first memory 113.

The first system is coupled to the second system 121 by a network 117. In some embodiments the network comprises the Internet.

The second system includes a second memory 123 and a second computer 125, including at least one processor. The second memory 123 stores private data, for example of one more individuals, who, in some embodiments, are the same, overlap, or are a subset of the individuals for whom EHR is stored in the first memory of the first system. In some embodiments the private data is stored in memory that may not generally be considered an integral part of the first system, for example in some embodiments the second memory may be that of a USB memory stick or other portable device including memory that may be read or provided to the second computer. The private data may derive from external sources such as social media, individual's consumer electronics, fitness tracking devices, or other personal monitoring or measuring devices.

The private data may be generated by the individual to whom the private data pertains, or may be generated by a care provider, for example a therapist, or by family members, or other sources such as social media or consumer electronic devices such as fitness trackers or applications or programs running on individual operated computers (e.g. an App on a cell phone). In some embodiments, for example, the second system may be a system of the therapist. The private data in many embodiments includes data pertaining to the individual that is not duplicated in the EHR of the first system, and in some embodiments the private data may differ from or contradict information of the EHR of the first system. In some embodiments the private data stored in the second system is not provided to the first system by the second system, and in some embodiments the private data stored in the second system is not considered to be EHR.

In some embodiments the second computer is configured to execute program instructions of a rules engine, which may the same as the rules engine of the first system or which may operate using different rules than the rules engine of the first system.

In operation, in some embodiments, the first system generates one or more health-related recommendations for one more individuals. The health-related recommendations are determined by the rules engine, based on information of the EHR. The first system also transmits the health-related recommendations and/or the EHR for a particular individual over the network to the second system. In doing so, the EHR for the particular individual may identify the second system, and/or the first and/or second systems may utilize various APIs, HL7 messages, a Health Information Exchange (HIE), or other method for establishing communications and/or transmitting the EHR. In some embodiments only a portion of the EHR for the particular individual may be transmitted, and in some embodiments only the health-related recommendation may be transmitted.

Also in operation, in some embodiments the second system generates one or more health-related recommendations for the particular individual. The health-related recommendations generated by the second system may be determined by the rules engine of the second system, based on the private data and, in some embodiments, the EHR provided by the first system. In some embodiments the second system additionally meshes or standardizes information of the DIR. For example, at times the EHR and the private data may be contradictory in some aspects, in which case the private data may be used instead of the EHR, or an option may be presented to a user of the second system to select which of the EHR or private data to have utilized by the rules engine.

In some embodiments the health-related recommendations generated by the second system are transmitted to the first system, so that a doctor associated with the first system may be aware of recommendations that have been or should be provided to the particular individual. In some embodiments, however, the second system never transmits to the first system the health-related recommendations generated by the second system. By not providing such recommendations to the first system, those recommendations may not become part of the EHR for the first system, for example. In some embodiments, a therapist or other individual or entity associated with the second system may provide the recommendations of the second system, and the recommendations of the first system in some embodiments, to the particular individual. The particular individual may then decide whether to follow the first recommendation, the second recommendation, to share the second recommendation with the medical professional of the first system, to consult with a 3rd medical professional, to do nothing, or to make their own decisions about their own health utilizing or not utilizing either recommendation.

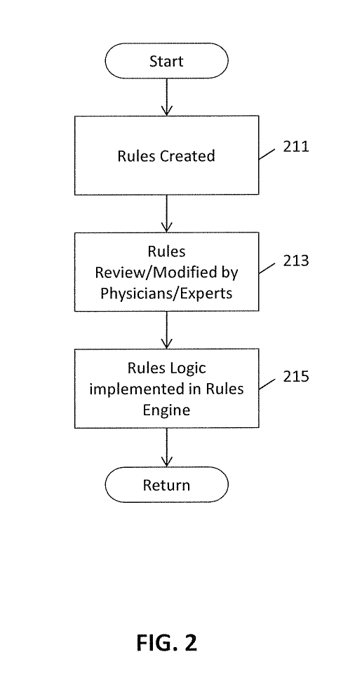

FIG. 2 is a flow diagram of a process for creating a rules engine, for example for a CDS. In block 211 rules are created. Rules define logic for recommending treatment or advice based on individual information or data. The rules may be created by physicians or other professionals. Rules may be entered into a computer format based on medical or health reference material, such as research papers or medical or pharmaceutical databases.

In some embodiments the rules define logic for combining data. Data about individuals may be inconsistent, incomplete, or contradictory. For example, an individual's name might be spelled differently from different sources. In the same source information that is entered more than once might not be consistent. For example, a patient might describe their medical history differently to different doctors entering data in the same EHR. Data may be incomplete and need to be combined. For example, a patient might have had three previous surgeries, but the one record may describe only one of them, while another record describes the other two. Information about an individual change over time. For example, a patient might gain weight, in which case the latest data would be used by the rules engine. Data may be incorrect due to human error, or changes in the patient's care. For example a patient may visit a first doctor and receive a prescription A. A visit to a second doctor and the prescription is changed from A to B. The EHR for the first doctor would describe prescription A, but not B. Information may also sometimes combined. One set of records may not include the use by an individual of over-the-counter vitamins and supplements, while another does.

In block 213 the rules are reviewed by physicians or other medical licensed professionals. In the case of rules that follow visible logic, the rules may be directly reviewed by physicians. Some rules engines use machine learning to adapt or improve the rules over time or over use. In this case the updated rules are reviewed from time to time by competent technical professionals and medical professionals. In all embodiments the rules may be reviewed by a medical professional to comply with laws, regulations, or professional guidelines regarding the implementation of CDS. The rules may be reviewed or modified by individual physicians for their own practice of medicine. Rules may be reviewed by physicians responsible for reviewing rules for a group of physicians, such as a medical group, a hospital, a pharmacy, or an insurance company.

Rules generally use an input data about an individual. This individual data may be considered part of the individual medical record. It may be stored in an EHR, but not considered part of a medical record. It may be data that can come from either an EHR or from private data. It may be data that only comes from private data.

In block 215 the rules logic is implemented in a rules engine. A rules engine may be a computer program that takes individual patient information, applies the logic of the rules engine, and outputs a recommendation, or a set of recommendations. The rules of a rules engine may be static, in that they are determined ahead of time and do not change without human intervention. The rules of a rules engine may change based on their own internal rules as in machine learning.

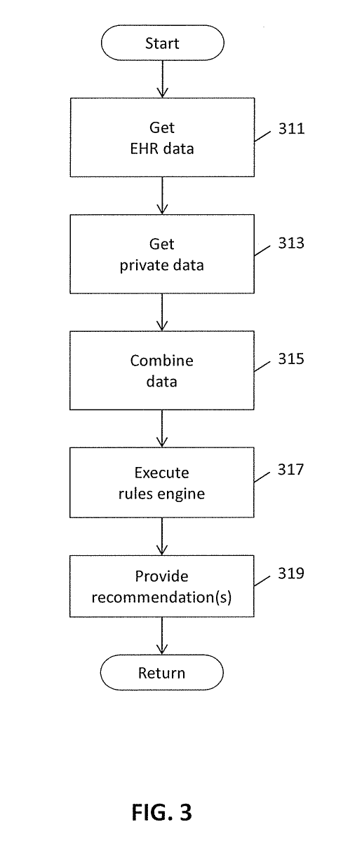

FIG. 3 is a flow diagram of a process for determining a health-related recommendation using EHR and private data in accordance with aspects of the invention. In some embodiments the process of FIG. 3 is performed by a system, for example the system of FIG. 1. In some embodiments the process of FIG. 3 is performed by a system such as the second system of FIG. 1. In some embodiments the process of FIG. 3 is performed by a processor, for example a processor executing program instructions. In some embodiments the process of FIG. 3 may be considered a process for a rules engine to apply logic to information about an individual, including private information not part of an EHR, for the purpose of determining one or more health-related recommendations for that individual.

In block 311 the process receives data from an EHR. In some embodiments a computer system receives the data from another computer system storing the EHR. In some embodiments the data is EHR for a particular individual. In some embodiments the data comprises a health-related recommendation for the particular individual. In some embodiments the data instead or in addition comprises EHR data related to the health-related recommendation for the individual.

In block 313 the process receives private data for the particular individual. In some embodiments the computer system receives the data from memory associated with the computer system. In some embodiments the computer system receives the data from a portable device including memory, for example a USB device or smartphone of the particular individual.

In optional block 315, the process combines the EHR data and the private data. In some embodiments the data is combined according to rules of a rules engine. Combined data may consist of data with duplicate data removed, incomplete data combined, or contradictory data resolved. In some embodiments data contradictions are resolved by presenting an option to select data from the HER or data from the private data. In some embodiments data contradictions are resolved by selecting the private data instead of the contradictory EHR data.

In block 316 the process executes the rules engine. In some embodiments the rules engine is as discussed with respect to FIG. 1, or elsewhere. In some embodiments execution of the rules engine results in zero, one, or more health-related recommendations based on logic of the rules engine.

In block 319 the process provides the health-related recommendations. In some embodiments the recommendations are provided to a computer system associated with a doctor or medical entity, for example by way of being transmitted over a network. In some embodiments the recommendations are output in a digital, visual, audio, or printed format. In some embodiments the recommendations may be comprised of instructions, data, or configurations for other EHRs, Medical Devices, consumer electronics, or may be human readable instructions. In some embodiments the recommendations may comprise or consist of health advice. In some embodiments the recommendations may comprise or consist of prescriptions for drugs, procedures, equipment, or the use of commercial exercise equipment. In some embodiments the recommendations may comprise or consist of the use of Apps for exercise or meditation. In some embodiments the recommendations are provided to one, some, or all of the individual, their caregiver/parent, the therapist, or the physician for the individual.

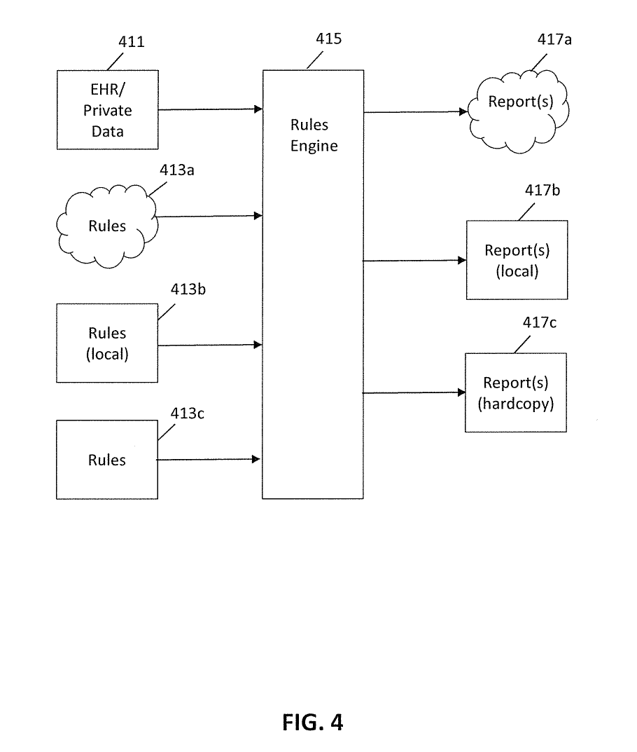

FIG. 4 is a block diagram showing data flow for a process for determining a health related recommendation using EHR and private data in accordance with aspects of the invention. In some embodiments the block diagram is a block diagram of portions of a system for performing operations of the process of the flow diagram of FIG. 3.

In FIG. 4, a rules engine 415 receives EHR and private data 411. The rules engine may be as discussed herein, and the rules engine may execute, for example as program instructions, on a computer system. In some embodiments the rules engine comprises logic describing condition on which recommendations will be output, and in some embodiments program instructions that executes that logic. In some embodiments rules 413b for the rules engine are locally stored by the computer system. In some embodiments rules 413a are received over a network, for example comprising the Internet. In some embodiments rules 413c are otherwise provided. The computer system may be the computer system of a mental health professional, in some embodiments. In some embodiments the EHR and the private data is combined, for example as discussed with respect to the process of FIG. 3. In some embodiments the private data includes information not found in the EHR, or contradictory to the information of the EHR.

The rules engine generates health-related recommendations. In some embodiments the rules engine generates recommendations 417a for provision over a network, for example comprising the Internet. In some embodiments the rules engine generates recommendations to be stored locally by the computer system. In some embodiments the rules engine generates recommendations to be provided in hardcopy form to or for the individual to whom the health-based recommendations pertain. In some embodiments the recommendations are in the form of reports. In some cases the health-related recommendations are electronic data for configuring medical devices or consumer electronic devices.

Although the invention has been discussed with respect to various embodiments, it should be recognized that the invention comprises the novel and non-obvious claims supported by this disclosure.

What is claimed is:

1. A system for determining health-related recommendations, comprising:
a first memory storing electronic health care records (EHR) for a plurality of individuals;
a first processor coupled to the first memory, the first processor configured to execute instructions of a rules engine associated with the first processor for determining a health-related recommendation for a particular individual of the plurality of individuals based on at least some of the EHR;
at least one second memory storing private information for the particular individual of the plurality of individuals, the at least one second memory not readable by the first processor; and
a second processor coupled to the second memory, the second processor configured to request and receive from the first processor the EHR for the particular individual and to execute instructions of a rules engine associated with the second processor for determining a health-related recommendation based on at least some of the EHR and at least some of the private information for the particular individual.

2. A method for use in providing health-related recommendations, comprising:
storing electronic health care records (EHR) for a plurality of individuals;
executing instructions, by a first system, of a rules engine associated with the first system for determining a first health-related recommendation for a particular individual based on at least some the EHR;
transmitting at least some of the EHR for the particular individual to a third party system, the third party system storing private information for the particular individual, at least some of the private information not being information included in the EHR; and
executing instructions, by the third party system, of a rules engine associated with the third party system for determining a second health-related recommendation for the particular individual based on at least some the EHR and at least some of the private information for the particular information.

3. The method of claim 2 further comprising transmitting information of the second health-related recommendation to the first system.

4. The method of claim 2 further comprising providing information of the first health-related recommendation and the second health-related recommendation to the particular individual, without providing information of the second health-related recommendation to the first system.

5. The method of claim 2, wherein the at least some of the EHR transmitted to the third party system comprises information of the first health-related recommendation. In some embodiments the method further comprises comparing, by the third party system, information of the first health-related recommendation and the second health-related recommendation.

6. The method of claim 2, wherein the EHR for the particular individual includes an identification of the third party system.

7. The method of claim 2, wherein the EHR for the particular individual does not include an identification of the third party system.

8. The method of claim 7, wherein the EHR for the particular individual includes a key for use in determining if a third party requestor should be allowed access to the EHR for the particular individual.

9. The method of claim 2, wherein the rules engine associated with the first processor and the rules engine associated with the third party system are the same rules engine.

10. The method of claim 2, wherein the rules engine associated with the first processor and the rules engine associated with the third party system implement the same rules.

11. The method of claim 2, wherein the rules engine associated with the first processor and the rules engine associated with the third party system implement different rules.

12. A method for use in providing health-related recommendations, comprising:
storing electronic health care records (EHR) for a plurality of individuals;
executing instructions, by a first system, of a rules engine for determining a first health-related recommendation for a particular individual based on at least some the EHR;
transmitting options for the first health-related recommendation to a third party system, the third party system storing private information for the particular individual, at least some of the private information not being information included in the EHR; and
executing instructions, by the third party system, of the rules engine based on at least some of the private information to determine whether options for the first health-related recommendation are contraindicated or re-prioritized by the private information.

13. The method of claim 12 further comprising determining that at least some options for the first treatment program are contraindicated or reprioritized by the private information.

14. The method of claim 12, further comprising: transmitting results of the determination whether options for the first treatment program are contraindicated by the private information to the first system.
